(19) United States
(12) Patent Application Publication
(10) Pub. No.: US 2024/0219407 A1
(43) Pub. Date: Jul. 4, 2024

(54) HAPTOGLOBIN DRY STICK

(71) Applicant: DELAVAL HOLDING AB, Tumba (SE)

(72) Inventors: John M CHRISTENSEN, Tumba (SE); Claus DALLERUP RASMUSSEN, Tumba (SE); Jonas TRIER HALD, Tumba (SE)

(21) Appl. No.: 18/563,270

(22) PCT Filed: May 18, 2022
(86) PCT No.: PCT/SE2022/050483

§ 371 Date: Nov. 21, 2023

(30) Foreign Application Priority Data
May 24, 2021 (SE) 2150654-8

(51) Int. Cl. G01N 33/72; G01N 33/543

(52) U.S. Cl. CPC G01N 33/72; G01N 33/54388; G01N 2800/365

(57) ABSTRACT

A dry stick being a competitive lateral flow stick configured to measure haptoglobin in a raw milk sample, the dry stick having a base pad that allows lateral flow of fluid therethrough, with a labelled-control conjugate and a labelled-conjugate diffusibly arranged herein, the labelled-conjugate binding haptoglobin, a complex forming between the labelled-conjugate and the haptoglobin. The base pad also includes a test line with immobilised target analyte, the immobilised target analyte binding to the labelled-conjugate when not in the complex; and a control line, which is spaced from the test line, and which includes control analyte that binds to the labelled-control conjugate.

### FIELD OF THE INVENTION

The present invention relates to a dry stick for measuring haptoglobin in a raw milk sample, where the dry stick is a lateral flow stick. The present invention further relates to a method of measuring haptoglobin in a raw milk sample.

### BACKGROUND OF THE INVENTION

Milk production in animals, especially cows have been optimized severely over the years through efficiency gain and selective breeding. The higher yield requires more feed but also healthy cows and good farm management to be maintained. Until now the industry has mainly been focusing on selective breeding and genetic breeding in relation to milk yield. However, increasing focus on disease detection and treatments is starting to be of more interest. Monitoring of parameters in the milk from cows could be of support hereof.

Several diseases may affect the milk yield and health status of the cows such as mastitis acute and chronic, ketosis and metabolic disorders, metritis and other reproductive diseases, stress and lameness. Mastitis is a common disease which is an infection in the mammary gland. The consequences are low milk yield and quality. Treatments are based on antibiotics and can be with limited success. Ketosis is a metabolic disorder, which happens the first months after calving. This is often treated with propylene glycol and feed containing high amount of starch (and other sugar derivatives). Metritis is an infection in the uterine. Happens after calving, the effect is low success rate on inseminations, increasing the open days, which in return extends that time with low milk yield, before the next calving time. Stress affect the well-being of the cow and can give abortions, low milk yield and low success rate of inseminations. Lameness is often caused by laminitis, claw disease, digital dermatitis, and foot rot. This limits the cows ability to move around and hereby access to feed, water and the general welfare of the cow. Diseases such as metritis, mastitis, lameness and ketosis can be so severe that the outcome is that the cow is set to be culled. Almost all diseases impact profitability on the farm and animals welfare. Optimal treatments and prevention methods for the above-mentioned diseases is thus, of high importance.

For a long time it has been generally known that early detection of diseases increases the chances of effective treatment. Therefore, there is an incentive to increase on farm monitoring and testing. To do this many systems have been developed. Automatic health and reproduction monitoring are emerging to improve efficiency, labor use and animal welfare in a sustainable way.

The mentioned diseases trigger an early defense response of the innate immune system affecting the expression of several proteins. One of these is the acute phase protein haptoglobin. Haptoglobin is positively upregulated as a cause of infections and is generally low in healthy cows why it constitutes a good disease marker as a response to infection and inflammation. Haptoglobins main biological function is to bind free hemoglobin with high affinity to prevent loss of iron after intravascular hemolysis. Haptoglobin is present 24-48 hours after infection and has been demonstrated to be present in milk. Haptoglobin can thus be used as a general health marker and for detection of diseases such as acute mastitis, chronic mastitis, metritis, lameness and respiratory diseases. Thus, haptoglobin could be a rapid, precise and easy diagnostic tool to evaluate the well-being of the cows and to establish the severity of potential diseases as well as a tool to screen the herd and point out animals for further health attention potentially followed by veterinarian assessment.

Hence, an easy available measurement system for potential on-site measurement of haptoglobin would be advantageous, and in particular, a reliable system for quantitatively measuring of haptoglobin to determine the present health status of milk-producing animals would be advantageous.

### OBJECT OF THE INVENTION

In particular, it may be seen as an object of the present invention to provide a dry stick and method for easy and reliable measurement of haptoglobin in raw milk that solves the above mentioned problems and provides the farmer with a readily available tool for establishing the health conditions of his animals.

### SUMMARY OF THE INVENTION

Thus, the above described object and several other objects are intended to be obtained in a first aspect of the invention by providing a dry stick being a competitive lateral flow stick configured to measure haptoglobin in a raw milk sample, said dry stick comprises:
a base pad capable of allowing lateral flow of fluid there through;
   comprising a labelled-control conjugate and a labelled-conjugate diffusibly arranged herein, wherein said labelled-conjugate binds haptoglobin, and wherein a complex is formed between said labelled-conjugate and said haptoglobin when said dry stick is in use;

wherein said base pad further comprises
a test line comprising immobilised target analyte, wherein said immobilised target analyte binds to said labelled-conjugate when not in said complex; and
a control line, which is spaced from said test line, and which comprises control analyte capable of binding to said labelled-control conjugate.

The invention is particularly, but not exclusively, advantageous for obtaining a direct measurement of haptoglobin in raw milk, and thus to obtain a measurement of the milk on the farm.

Another aspect of the invention relates to providing a dry stick being a competitive lateral flow stick configured to measure haptoglobin in a raw milk sample, said dry stick comprises:
a base pad capable of allowing lateral flow of fluid there through;
   comprising a labelled-control conjugate and a labelled-target analyte diffusibly arranged herein;

wherein said base pad further comprises
a test line comprising an immobilised binding molecule, said immobilised binding molecule binds to haptoglobin or said labelled-target analyte; and
a control line, which is spaced from said test line, and which comprises a control analyte capable of binding to said labelled-control conjugate.

A further aspect of the invention relates to providing a method of measuring haptoglobin in a raw milk sample
a) providing an raw milk sample potentially comprising haptoglobin;
b) providing a dry stick as described herein comprising a test line and a control line;
c) applying said raw milk sample to said dry stick;
d) allowing said raw milk sample to flow through said test line and said control line on said dry stick;
e) determining the amount of haptoglobin.

This aspect of the invention is particularly, but not exclusively, advantageous in that the method according to the present invention may be implemented on the farm for quick determination of potentially sick animals.

The aspects of the present invention may each be combined with any of the other aspects and embodiments. These and other aspects of the invention will be apparent from and elucidated with reference to the embodiments described hereinafter.

### BRIEF DESCRIPTION OF THE FIGURES

The dry stick according to the invention will now be described in more detail with regard to the accompanying figures. The figures show one way of implementing the present invention and is not to be construed as being limiting to other possible embodiments falling within the scope of the attached claim set.

FIG. 1 shows an embodiment of the dry stick according to the invention. A) is a side view of the dry stick, while B) is a top view of the dry stick;

FIG. 2 shows the principle of the dry stick according to two aspects of the invention; A-C) show the principle according to one embodiment of the invention, while D-F) show the principle according to another embodiment of the invention;

FIG. 3 shows the measurements of the test line and control line on an embodiment of a dry stick;

FIG. 4 shows the average performance of a dry stick on samples spiked from 0-10 μg/ml with haptoglobin, for an independent versus dependent control line set-up. x-axis: concentration of haptoglobin;

FIG. 5 shows the average performance of the dry stick on samples spiked from 0-20 μg/ml with haptoglobin. The dose curve is close to linear for the ratio R, R=test signal/(test signal+control signal). x-axis: concentration of haptoglobin;

FIG. 6 shows a mathematical model used for the calculation of the amount of haptoglobin (HP) measured in raw milk samples. x-axis: concentration of haptoglobin (HP);

FIG. 7 schematically illustrates an embodiment of a milking arrangement in which a dry stick according to the invention may be used.

The present invention will be described in more detail in the following.

### DETAILED DISCLOSURE OF THE PRESENT INVENTION

The inventors of the present invention surprisingly discovered that a dry stick could be developed, which were able to measure the level of haptoglobin in raw milk in a quick, reliable and quantitative manner enabling the farmer without any problems to decide on the health status of his milk-producing animals and obtain a measurement of the severity of potential diseases. The dry stick may be used in commonly known milking arrangements for daily monitoring of the health status of the milk-producing animals. Hereby, early detection of potential severe diseases is possible, enabling early treatment and thus, the well-being of the cow and the milk yield may be improved.

### Dry Stick

In a first aspect, the present invention relates to a dry stick being a competitive lateral flow stick configured to measure haptoglobin in a raw milk sample, said dry stick comprises:
a base pad capable of allowing lateral flow of fluid there through;
   comprising a labelled-control conjugate and a labelled-conjugate diffusibly arranged herein, wherein said labelled-conjugate binds haptoglobin, and wherein a complex is formed between said labelled-conjugate and said haptoglobin when said dry stick is in use;

wherein said base pad further comprises
a test line comprising immobilised target analyte, wherein said immobilised target analyte binds to said labelled-conjugate when not in said complex; and
a control line, which is spaced from said test line, and which comprises control analyte capable of binding to said labelled-control conjugate.

By competitive lateral flow stick is to be understood that the dry stick is capable of allowing a lateral flow of liquid from one end of the dry stick to the other end of the dry stick as commonly known to the skilled person. By competitive is to be understood that the target analyte at the test line competes with haptoglobin in the sample for binding to the labelled-conjugate.

A dry stick as herein described may be implemented in a milking arrangement, potentially via a cassette, as described in e.g. WO 2020/251457.

### Base Pad

The dry stick comprises a base pad for allowing a lateral flow to take place. In its most simple form the base pad only comprises one module. However, the base pad may also comprise more modules, which can be of different materials depending on the specific purpose of the module. In an embodiment, said base pad comprises at least two modules. Hereby is to be understood that the base pad may comprise two or more modules.

In a further embodiment, the base pad comprises two modules being a membrane and a reagent pad. In a further embodiment, said membrane or at least a part hereof is downstream of said reagent pad.

In a further embodiment, the base pad comprises three modules being a membrane, a sample pad and a conjugate pad. In a further embodiment, said membrane or at least a part hereof is downstream of said conjugate pad and said conjugate pad or at least a part hereof is downstream of said sample pad.

In a further embodiment, a module is a reagent pad capable of receiving said raw milk sample. Hence, the base pad would comprise a module being a reagent pad. In an even further embodiment, said reagent pad comprises at least two modules being a sample pad and conjugate pad. In a further embodiment, the sample pad and the conjugate pad are partly overlapping. In one embodiment, the base pad comprises at least two modules, wherein one of said at least two modules is a sample pad capable of receiving said raw milk sample. If the reagent pad is one module, the sample pad and conjugate pad will be contained in this module and the raw milk sample will be received in the reagent pad.

In the present context, “reagent pad” relates to one or more pads comprising the labelled-control conjugate and the labelled-conjugate, which are both diffusible arranged in the reagent pad. The raw milk sample would also be applied to the reagent pad. In one embodiment, the reagent pad comprises a sample pad and a conjugate pad.

The material used for the reagent pad may be selected from the group of a nitrocellulose membrane, a cellulose, a polymer such as nylon, a polyvinylidene fluoride or latex, glass fibres, woven fibres, non-woven fibres and a chromatographic gel membrane.

In the present context, “sample pad” relates to a pad in the dry stick where the raw milk sample is applied to the dry stick and which provides a fast adsorption of the liquid sample and a fast and consistent release of the sample to the conjugate pad. The purpose of the sample pad is to collect sample. In some embodiments, it can be designed to withhold not wanted molecules present in the sample for these not to interfere with the functioning of the dry stick such as removing red blood cells, fat aggregates and large particles. The sample pad may be treated with a surfactant to release surface tension and quickly soak in the sample. The material used for the sample pad may be selected from the group of a nitrocellulose membrane, a cellulose, a polymer such as nylon, a polyvinylidene fluoride or latex, glass fibres, woven fibres, non-woven fibres and a chromatographic gel membrane.

In the present context, “conjugate pad” relates to one or more pads comprising the labelled-control conjugate and the labelled-conjugate, which are both diffusible arranged in the conjugate pad.

The material used for the conjugate pad may be selected from the group of a nitrocellulose membrane, a cellulose, a polymer such as nylon, a polyvinylidene fluoride or latex, glass fibres, woven fibres, non-woven fibres and a chromatographic gel membrane.

In the present context, “diffusibly arranged herein” relates to the labelled-control conjugate and the labelled-conjugate being present in the base pad in a manner, which allows the labelled-control conjugate and the labelled-conjugate to be immobilised when the dry stick is in dry state and mobile when in moistened state i.e. when in use. Accordingly, the labelled-conjugate and the labelled-control conjugate will be maintained in the base pad e.g. in the reagent pad or conjugate pad when the dry stick is not used. When sample is added to the dry stick, the induced flow will transfer the labelled-control conjugate and the labelled-conjugate along the flow.

In a further embodiment, said base pad comprises a membrane; said membrane comprising said test line and said control line. In a further embodiment, said membrane is a nitrocellulose membrane. In one embodiment, one of the modules of the base pad is a membrane such as a nitrocellulose membrane. The nitrocellulose membrane has a porous structure, which makes it suitable for migration of liquid through capillary action. The membrane comprises two lines—a test line and a control line. Flow rates along the dry stick may be controlled by the characteristics of the membrane.

In a still further embodiment, said dry stick further comprises an absorbent pad.

In the present context, the term “absorbent pad” refers to a material, which has the purpose of absorbing any liquid in excess when it has migrated through the base pad. Furthermore, backflow is prevented, which could cause incorrect results. Accordingly, the absorbent pad is arranged downstream of the test line and control line.

The material for the absorbent pad can be any material having great absorption characteristics such as a cellulose based material.

In an even further embodiment, said dry stick further comprises a backing card. In the present context, the term “backing card” refers to a material, which has no influence on the migration or on the reaction of the liquid sample or on reagent(s) or the agents capable of increasing the rate of the reaction. The backing card provides a stabilising basis for the dry stick and provides sufficient strength to maintain the desired physical shape and has substantially no interference with the production of a detectable signal. Thus, the backing card supports and stabilises at least a part of the base pad and potentially at least a part of the absorbent pad. In an embodiment of the present invention, the material for the backing card is selected from the group of polystyrene, vinyl and adhesive.

In a further embodiment, said dry stick further comprises a cover tape. In an even further embodiment, said cover tape covers at least said conjugated pad. In the present context, the term “cover tape” refers to a material, which has the purpose of making contact between the different membranes and/or pads. It has no chemical function but serves solely to apply pressure and contact between the different modules. The material could be any clear tape, where the adhesive does not have any influence flow of the dry stick.

In yet an embodiment of the present invention, the modules of the dry stick are in contact with one another by substantially fully overlapping, by partially overlapping or by laying adjacent to one another. In a further embodiment, said at least two modules partly overlaps. In an embodiment of the present invention the modules are overlapping by at least 5%, such as at least 10%, e.g. at least 25%, such as at least 50%, e.g. at least 75%, such as at least 80%, e.g. at least 90%, such as at least 95%. In the present context the term “substantially fully overlapping” relates to two separate modules being placed on top of one another.

In the present context the term “partially overlapping” relates to two separate modules being overlapping with only part of the modules. A partial overlap of 100% relates to a full overlap and a deviation of 5% from the 100% full overlap relates to a substantially full overlap.

In an embodiment of the present invention the modules are laying adjacent to one another. This means that the pads are placed in contact with each other (touching each other). An overlap of 0% (but in contact) relates to the term “laying adjacent”, furthermore, an overlap of less than 5% may be considered being within the term of “laying adjacent”, such as an overlap of at the most 4%, e.g. an overlap of the most 3%, such as an overlap of the most 2% or e.g. an overlap of the most 1%.

In a preferred embodiment, the dry stick comprises a backing card and a base pad having three modules being a sample pad, a conjugate pad and a membrane having a control line downstream of the test line. The sample pad partially overlaps with the conjugate pad, which partially overlaps with the membrane. The dry stick furthermore comprises an absorbent pad downstream of the membrane and partially overlapping herewith.

In a further preferred embodiment, the dry stick comprises a backing card and a base pad having two modules being a reagent pad and a membrane having a control line downstream of the test line. The reagent pad partially overlaps with the membrane. The dry stick furthermore comprises an absorbent pad downstream of the membrane and partially overlapping herewith.

### Test Line and Control Line

The test line comprises a target analyte capable of binding to the labelled-conjugate, when not bound to haptoglobin. The control line comprises a control analyte capable of binding to the labelled-control conjugate. Accordingly, the control line is independent of the test line. In one embodiment, said control line is downstream of said test line. This is highly advantageous as it follows from the independency that the control line is to ensure that the stick is functioning and thus, the result obtained in the test line valid. When arranging the control line downstream of the test line, the functioning is tested after the test line and thus, it follows that the flow in the dry stick is correct at least until the control line is reached.

The wording “line” refers to the area where the target analyte and control analyte, respectively, are immobilized on the dry stick. This is often in the shape of a line, however, it may also be formed in a different geometrical pattern as long as the read-out of the binding to the target analyte and control analyte is feasible.

In the present context, “control analyte” is to be understood as the compound immobilized at the control line, which is capable of binding to the labelled-control conjugate. The control analyte is an immobilised control analyte. Hereby, is to be understood that the control analyte will remain at the control line even when said dry stick is in use.

The control analyte may be selected from the group of monoclonal antibodies, polyclonal antibodies, chimeric antibodies, nanobodies, aptamers and antibody mimicking proteins. In an even further embodiment, said control analyte is a third antibody.

In one embodiment, the concentration of the control analyte at the control line is 0.01-2 mg/ml, such as 0.02-1 mg/ml, like 0.05-0.5 mg/ml, such as around 0.2 mg/ml.

In the present context, “target analyte” is to be understood as the compound immobilized at the test line. The target analyte is capable of binding to the labelled-conjugate but not when the conjugate is in complex with haptoglobin. Accordingly, the target analyte may be haptoglobin or a part hereof as long as this part is able to bind to the labelled-conjugate in a competitive manner with the haptoglobin protein present in the raw milk sample. Alternatively, the target analyte may be a fusion protein comprising at least the part of haptoglobin binding to the labelled-conjugate. As an example, at least the part of haptoglobin binding to the labelled-conjugate or haptoglobin as such could be fused to bovine serum albumin (BSA). Alternatively, at least the part of haptoglobin may be biotinylated for immobilisation purposes.

The target analyte is an immobilised target analyte. Hereby, is to be understood that the target analyte will remain at the test line even when said dry stick is in use.

In one embodiment, the concentration of the target analyte at the test line is 0.01-2 mg/ml, such as 0.02-1 mg/ml, like 0.05-0.5 mg/ml, such as around 0.1 mg/ml.

In the present context, “complex” relates to a molecular entity formed by association between the labelled-conjugate in the base pad and the haptoglobin potentially present in the raw milk sample, which will form when the dry stick is in use i.e. when a sample is added to the dry stick and said sample comprises haptoglobin.

In one embodiment, the width of the control line and/or test line is 0.5-5 mm, such as 1-3 mm, like around 2 mm and the distance between the lines would be at least 0.5 mm, such as at least 1 mm in order to allow for sufficient distance between the lines. This would also allow for measurement of a background between the test line and control line to enable a more reliable measurement of the output of the test line and control line.

In a further embodiment, the target analyte and/or control analyte may be immobilized on the test line and/or control line, respectively by using blocking compounds such as polyvinylpyrrolidone (PVP), polyethylene glycol (PEG), bovine serum albumin (BSA) and salts like NaCl and KCl, to help bind the control analyte and/or target analyte to the base pad.

In addition, or alternatively, the target analyte and/or control analyte may be accompanied by pH-regulating agents such as Tris buffers, phosphate buffers and glycine buffers, for stabilising the pH and hence the secondary structure of the protein.

In a still further embodiment, surfactants may be included like Tween-20, Triton x-10, Plurionic, Tegretiol 20.

### Raw Milk Sample

In the present context, “raw milk sample” is to be understood as a milk sample obtained directly from the animal after milking, where the sample has not yet been processed to consumer milk.

The animal is preferably a cow, but the invention may be used for other milk producing animals. “Animal” may be any arbitrary type of domesticated female milk producing and/or meat producing mammal, such as cow, goat, sheep, horse, camel, primate, dairy buffalo, donkey, yak, etc.

Advantageously, the dry stick may comprise a surfactant for the raw milk sample to provide optimal mix, release and line morphology when running the raw milk samples. In one embodiment, said base pad further comprises a surfactant. In a further embodiment said sample pad comprises said surfactant. In an even further embodiment, said surfactant is comprised in said reagent pad. In particular, the use of a surfactant in connection with the raw milk sample is beneficial with respect to the measurement of haptoglobin as the viscosity of the raw milk is usually increased for animals suffering from a disease.

In one embodiment, said surfactant is Pluronic F68, Pluronic F127, Surfactant 10G, Synperonic F108, Tergitol, Tween-20 and/or Triton X-100.

In a further embodiment, said surfactant is in a concentration in the range of 0.1-5 w/w %, such as 0.5-4 w/w %, like 1-3 w/w %, such as around 2 w/w %.

In an even further embodiment, said base pad comprises a pH-regulating agent. In a still further embodiment, said pH-regulating agent is a phosphate buffer, a borate buffer, a citric acid buffer and/or a Tris buffer. The pH-regulating agent ensures that the pH is maintained in the raw milk sample around a pH resembling that of the raw milk sample. In one embodiment, said raw milk sample has a pH in the range of 6-9. In a further embodiment, the raw milk sample has a pH in the range of 6.4-8.5. Hereby, the confirmation of the proteins in the raw milk sample will maintain as naturally occurring and allow for optimal binding of haptoglobin to the labelled-conjugate and/or optimal binding of the target analyte to the labelled-conjugate. Accordingly, pH is maintained optimal during the use of the dry stick.

In addition, further components may be added to the base pad and in particular to the conjugate pad in order to obtain a better release of the conjugate and the control-conjugate and validity of the results obtained when using the dry stick. Such compounds may be Sucrose and Trehalose. In one embodiment, these compounds are added in a concentration of 0.1-5 w/w %, such as 0.5-4 w/w %, like 1-3 w/w %, such as around 2 w/w % each.

Alternative or additionally to adding a surfactant to the base pad, sample pad and/or reagent pad, the raw milk sample may be mixed with a surfactant before providing it to the dry stick. In a further embodiment, said raw milk sample is mixed with a diluent comprising a surfactant, prior to applying said raw milk sample to said dry stick. In an even further embodiment, said surfactant is present in the diluent in a concentration of 0.1-4 w/w %, such as 0.5-2 w/w %, like around 1 w/w %.

In a still further embodiment, said surfactant is one or more surfactants selected from the following group: Pluronic F68, Pluronic F127, Surfactant 10G, Synperonic F108, Tergitol, Tween-20 and/or Triton X-100. In a still further embodiment, said surfactant is Tween-20.

Advantageously, the addition of a diluent increases the flowrate and mixing properties of the sample.

The diluent may beside water and surfactant comprise a pH regulating agent e.g. Tris-(hydroxymethyl)-methylamine), stabilizers e.g. sodium chloride, blocking agents e.g. PVP 40 and conservation agents e.g. ProClin 300. This would increase the reproducibility e.g. by blocking non-specific binding. Other pH regulating agents, stabilizers, blocking agents and conservation agents as commonly known to the skilled person may also be used.

The pH-regulating agent may be provided with the diluent. The concentration of the pH-regulating agent in the diluent is preferably in the range of 0.01-0.99 w/w %, such as 0.1-0.9 w/w %, like 0.25-0.75 w/w %, such as 0.35-0.65 w/w %, like around 0.5 w/w %.

The mixing of the diluent and the raw milk sample is preferably done in a sample: diluent ratio of 0.25:0.75-0.75:0.25, like 0.30:0.70-0.70:0.30, such as 0.35:0.65-0.65:0.35, like 0.40:0.60-0.60:0.40, such as 0.45:0.55-0.55:0.45, like around 0.50:0.50. In one embodiment, the sample: diluent ratio is 0.45-0.55.

### Conjugate

In the present context, “labelled-conjugate” relates to the chemical substance that binds to haptoglobin in the raw milk or to the target analyte on the test line. The labelled-conjugate is a conjugate labelled with a label. Labelled-conjugate may relate to different labelled-conjugates such as two or more labelled-conjugates, such as three or more labelled-conjugates. However, in one embodiment, only one labelled-conjugate is present in the dry stick. The labelled conjugate may comprise a conjugate being selected from the group of monoclonal antibodies, polyclonal antibodies, chimeric antibodies, nanobodies, aptamers and antibody mimicking proteins. In one embodiment, said labelled-conjugate is a labelled first antibody.

In one embodiment, the concentration of the labelled-conjugate is 0.5-20 μg/ml, such as 1-15 μg/ml, like 2-12 μg/ml, such as 3-10 μg/ml, like 4-7 μg/ml, such as around 5 μg/ml.

In the present context, “labelled-control conjugate” relates to the chemical substance that binds to the control analyte at the control line. The labelled-control conjugate is a control conjugate labelled with a label. Labelled-control conjugate may relate to different labelled-control conjugates such as two or more labelled-control conjugates, such as three or more labelled-control conjugates. However, in one embodiment, only one labelled-control conjugate is present in the dry stick. The labelled-control conjugate may comprise a control conjugate being selected from the group of monoclonal antibodies, polyclonal antibodies, chimeric antibodies, nanobodies, aptamers and antibody mimicking proteins. In a further embodiment, said labelled-control conjugate is a labelled second antibody.

The concentration of the control analyte in the control line is 1-30 μg/ml, such as 2-25 μg/ml, like 4-20 μg/ml, such as 5-15 μg/ml, like 7-12 μg/ml, such as around 10 g/ml.

The labelled-control conjugate and the labelled-conjugate may be added to the conjugate pad by soaking, dip coating and spraying as commonly known to the skilled person in the art. This may be performed either manually or automatically.

The labelled-control conjugate and the labelled-conjugate are labelled with a label in order to be identified at the control line and the test line. The label would depend on the measuring system for the measurement of the labelled-control conjugate and labelled-conjugate present at the control line and test line, respectively. Thus, the labels may be chosen by the skilled person according to the wishes of the read-out. The label may be selected from the group of colloidal gold particles, latex particles, cellulose nanobeads, paramagnetic particles, radioactive particles and fluorescent particles as well as enzymes. In one embodiment, said labelled-control conjugate and/or said labelled-conjugate is labelled with colloidal gold particles.

Standardly, the three major groups of labels are: gold nanoparticles, latex particles and fluorescent particles.

The gold particles are gold particles often with a diameter around 40 nm. The particles are often called colloidal gold particles, since they are in a stable dispersion and require a specific pH and additives to the dispersion. These particles have a reddish/purple color. The small size gives a quick release and less issues with aggregation. However, sensitivity is often lower, since the color formation is lower per conjugate and thereby requires more conjugates. This is due to the smaller size and thereby the less reflection surface. The conjugation of conjugates to the gold particles is done passively, meaning it utilizes a combination of the electrostatic surface of the colloidal gold and hydrophobic interactions to bind the protein to the gold particles. As an example, it may be performed by mixing the conjugate and gold particles at a specified pH, adding excess bovine serum albumine and removing all unbound conjugate by centrifugation.

Latex particles are microspheres made of polyesters. These have a size of about 200-400 nm in diameter. They can be made in a variety of colors such as blue, green, purple and red. The particles are larger meaning less conjugate is required to create a visual color than for gold particles. Thus, less conjugate would be needed per dry stick, which could result in more variation and less flexibility. The bigger size may cause issues in relation to release and aggregation. The conjugate and label is normally covalently conjugated.

Fluorescent particles give high sensitivity and detect analytes at very low concentrations (pg/mL). A disadvantage is that it requires a specific light source for reading of the result but the advantages are improved sensitivity. There are multiple different types of fluorescent labels, such as organic dyes, metal-ligand complexes, fluorescent proteins, semiconductor quantum dots, lanthanide complexes, dye-doped polymer nanoparticles, fluorescent silica nanoparticles, xanthene dyes and cyanine dyes. In addition, fluorescence proteins can also be utilized. These different types of fluorescent dyes have different absorbance and emission wavelengths why it may be used for multiplexing.

The labels may be conjugated to the conjugate and/or control conjugate either passively or covalently.

### Method of Measuring Haptoglobin

In a further aspect, the present invention provides a method of measuring haptoglobin in a raw milk sample
a) providing an raw milk sample potentially comprising haptoglobin;
b) providing a dry stick as described herein comprising a test line and a control line;
c) applying said raw milk sample to said dry stick;
d) allowing said raw milk sample to flow through said test line and said control line on said dry stick;
e) determining the amount of haptoglobin.

In the present context, “the amount of haptoglobin” is to be understood as an exact amount of haptoglobin, a concentration of haptoglobin or level of haptoglobin. Hereby, is to be understood that a quantitative measurement can be obtained and not just a qualitative measurement as is commonly seen in the art. It is highly advantageous that even low amounts of haptoglobin may be determined as this is able to provide the farmer with an indication of the severity of potential diseases of the animal and enables her to distinguish between a mild, a moderate and a severe disease.

In one embodiment, said raw milk sample is provided in a defined amount. This would be needed if the exact amount of haptoglobin present in the milk is to be determined.

In a further embodiment, said amount of haptoglobin is determined by measuring a test signal at said test line and a control signal at said control line, and calculating a signal ratio by dividing said test signal with the sum of said test signal and said control signal, and following using said signal ratio to retrieve said amount of haptoglobin from a look-up table or by applying a mathematical model. The signal ratio is a relative measurement between the test signal and the control signal. It has been found that inter alia aging of a dry stick may influence the colour intensity produced by the test line and, if implemented, also the colour intensity of the control line. Aging refers to the storage time of the dry stick and under which condition the dry stick has been stored. The colouring of the test line and control line is the result of the binding of the labelled-conjugate and labelled-control conjugate to the lines and the colour intensity is correlated with the amount of labelled conjugate and labelled-control conjugate, which binds to the control line and test line respectively. Without being bound by theory, it is suggested that less labelled conjugate and less labelled-control conjugate are released with increasing time. In attempt to at least mitigate such aging effects, the invention suggests to use a ratio R between test signal and control signal as: R=test signal/(test signal+control signal). This ratio R is in such cases correlated with the value of haptoglobin quantity and is found to be essentially independent on the amounts of labelled conjugate and labelled-control conjugate released from the base pad by the milk applied to dry stick.

The calculated signal ratio may be compared to a look-up table or a mathematical model in order to obtain the amount of haptoglobin, determined as the concentration of haptoglobin in the raw milk sample. This concentration would then be an indication of potential diseases in the animal as well as the severity of the diseases.

The term “look-up table” refers to a table comprising values for the dry stick in which a given ratio can be looked up and the corresponding amount of haptoglobin found. The term “mathematical model” refers to a model or formula, which can be used to calculate the amount of haptoglobin once the ratio for a given measurement on the dry stick is known. It follows that both the look-up table and the mathematical model is based on multiple experiments where a sample comprising known amounts of haptoglobin is added to the dry stick, and the corresponding ratio calculated based on measured control and test signals.

The measurement of the control signal and test signal may be performed by methods and systems known by the persons skilled in the art. The measurement method would furthermore depend on the labels of the conjugates as this would require different measurement techniques whether the label was e.g. a gold, latex or a fluorescent label.

In one embodiment, the measurement method takes into account the background obtained from the dry stick outside the test line and control line, preferably this background is measured in an area between the test line and the control line.

In a further embodiment, said test signal and/or control signal is measured using an optical device. In a still further embodiment, said optical device is a camera. In one embodiment, the test signal and/or control signal may be measured as described in WO 02/069697.

### Immobilised Binding Molecule and Labelled-Target Analyte

A further aspect of the present invention relates to a dry stick being a competitive lateral flow stick configured to measure haptoglobin in a raw milk sample, said dry stick comprises:
a base pad capable of allowing lateral flow of fluid there through;
   comprising a labelled-control conjugate and a labelled-target analyte diffusibly arranged herein;

wherein said base pad further comprises
a test line comprising an immobilised binding molecule, said immobilised binding molecule binds to haptoglobin and/or said labelled-target analyte; and
a control line, which is spaced from said test line, and which comprises a control analyte capable of binding to said labelled-control conjugate.

In this aspect, the dry stick may be any of the embodiments as described herein but having a labelled-target analyte instead of a labelled-conjugate and having an immobilised binding molecule at the test line instead of immobilised target analyte. By competitive is thus to be understood for this aspect that the labelled-target analyte competes with haptoglobin in the sample for binding to the immobilised binding molecule at the test line.

Accordingly, for this aspect, “diffusibly arranged herein” relates to the labelled-control conjugate and the labelled-target analyte being present in the base pad in a manner, which allows the labelled-control conjugate and the labelled-target analyte to be immobilised when the dry stick is in dry state and mobile when in moistened state i.e. when in use. Accordingly, the labelled-target analyte and the labelled-control conjugate will be maintained in the base pad e.g. in the reagent pad or conjugate pad when the dry stick is not used. When sample is added to the dry stick, the induced flow will transfer the labelled-control conjugate and the labelled-target analyte along the flow.

In this aspect, the base pad comprises a labelled-target analyte. In the present context, “labelled-target analyte” relates to the chemical substance that binds to the test line in competition with haptoglobin present in the raw milk. The labelled-target analyte is a target analyte as described herein being labelled with a label. The label may be any label as described herein. In one embodiment, said labelled-target analyte is labelled haptoglobin.

In this aspect, the test line comprises an immobilised binding molecule. In the present context, “immobilised binding molecule” relates to the chemical substance that binds to haptoglobin in the raw milk or to the labelled-target analyte during the flow of the sample. The binding molecule may be selected from the group of monoclonal antibodies, polyclonal antibodies, chimeric antibodies, nanobodies, aptamers and antibody mimicking proteins.

The binding molecule is an immobilised binding molecule. Hereby, is to be understood that the binding molecule will remain at the test line even when said dry stick is in use. The immobilised binding molecule is a binding molecule being immobilised at the test line as described herein.

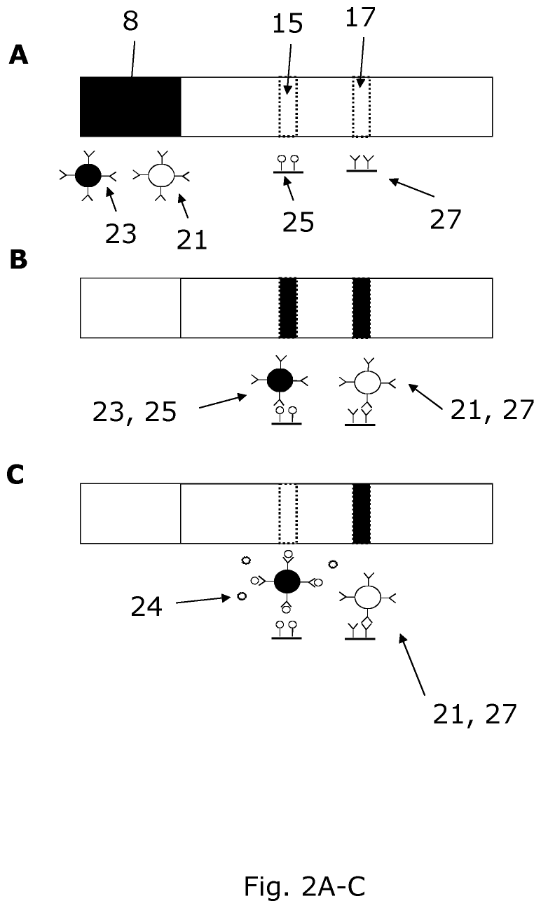

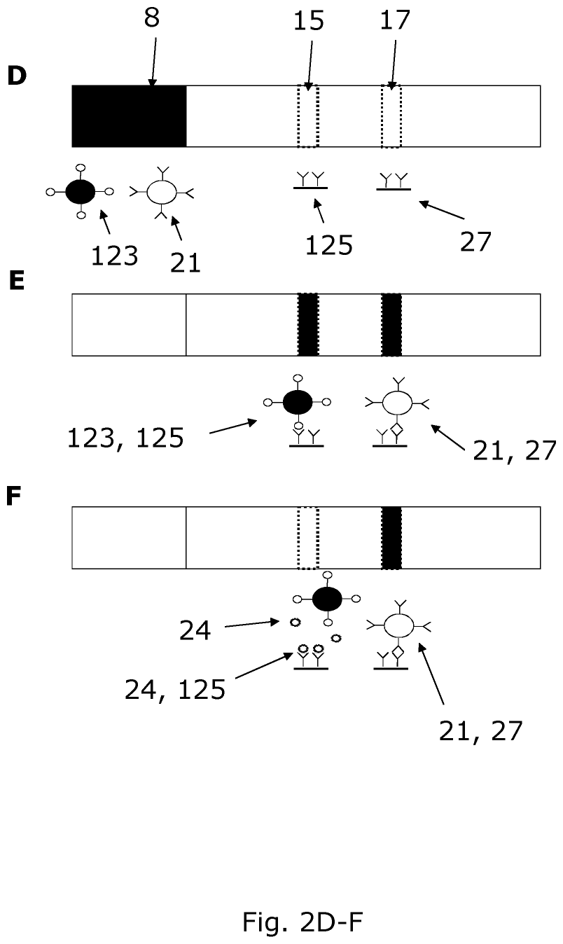

The use of an embodiment of this aspect is shown in FIG. 2D-F, illustrating the reaction in the dry stick in case of a raw milk sample containing haptoglobin (2F) or not containing haptoglobin (2E).

### EXAMPLES

### Example 1—Dry Stick for Haptoglobin Measurement

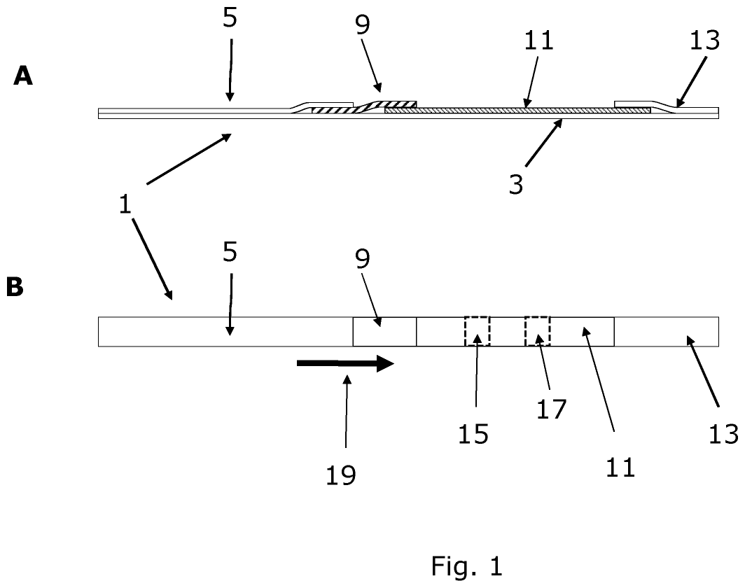

In one embodiment, the dry stick 1 may be designed as disclosed in FIG. 1A-B, where A shows a side view of the embodiment and B shows a top view. The dry stick 1 comprises a backing card 3 on which a sample pad 5, a conjugate pad 9, a membrane 11 and an absorbent pad 13 are arranged. Each of these different modules are partially overlapping to provide a smooth flow. The raw milk sample is added to the sample pad 5 and mixes with the conjugate (i.e. labelled-control conjugate and labelled-conjugate) in the conjugate pad 9 due to the downstream lateral flow 19 in the dry stick 1. Haptoglobin in the raw milk sample will bind to the conjugate to form a complex. Downstream of the conjugate pad 9 the membrane 11 comprises a test line 15 and a control line 17, which will bind to conjugate and control conjugate, respectively, from the conjugate pad 9. At the end of the run, the raw milk sample will be absorbed by the absorbent pad 13 at the end of the dry stick 1. Hereby, backflow is avoided along with contamination of the result. In addition, the dry stick will often hold a cover tape (not shown) to create pressure on all contact spots between the different materials and to ensure sufficient flow.

FIG. 2A-C show one embodiment of a dry stick prior according to the invention. FIG. 2A shows one embodiment of a dry stick prior to testing a raw milk sample. In this embodiment, the conjugate pad and the sample pad are shown as one module (i.e. reaction pad 8). The raw milk sample are introduced to the reaction pad 8, which comprises labelled-control conjugate 21 as well as labelled-conjugate 23. At the test line 15, target analyte 25 is present, while the control line 17 comprises control analyte 27.

If no haptoglobin is present in the raw milk sample, the labelled-conjugate 23 will bind to the target analyte 25 as demonstrated in FIG. 2B. In addition, the labelled-control conjugate 21 will bind to the control analyte 27. Hereby, a negative result is obtained being labelled both in the control line as well as the test line. In case of a positive result i.e. that haptoglobin 24 is present (shown in FIG. 2C) the haptoglobin will form a complex with the labelled-conjugate 23 prior to arriving at the test line 15 preventing the labelled-conjugate 23 from binding to the target analyte 25 at the test line 15. Nevertheless, the labelled-control conjugate will bind to the control analyte 27 at the control line 17. Thus, the dry stick will only be labelled at the control line 17. Depending on the concentration of haptoglobin in the raw milk, the labelled-conjugate 23 binds to the test line 15 correlating negatively (competitive) with the concentration haptoglobin. Accordingly, the intensity of the labelling at the test line 15 will vary depending on the amount of haptoglobin present in the raw milk sample.

The labelling at the control line 17 is independent of the amount of haptoglobin present in the raw milk sample and is a control of the lateral flow as such and the release of the conjugates from the conjugate pad. If the lateral flow has run satisfactorily and the conjugates been released as should be from the conjugate pad, a labelling will appear at the control line 17. If no labelling is registered at the control line 17, the result obtained by the dry stick cannot be trusted. It is thus advantageous for the control line to be present downstream of the test line for the results at the test line to be considered valid.

FIG. 2D-F show another embodiment of a dry stick prior according to the invention. FIG. 2D shows one embodiment of a dry stick prior to testing a raw milk sample. In this embodiment, the conjugate pad and the sample pad are shown as one module (i.e. reaction pad 8). The raw milk sample are introduced to the reaction pad 8, which comprises labelled-control conjugate 21 as well as labelled-target analyte 123. At the test line 15, binding molecule 125 is present, while the control line 17 comprises control analyte 27.

If no haptoglobin is present in the raw milk sample, the labelled-target analyte 123 will bind to the binding molecule 125 as demonstrated in FIG. 2E. In addition, the labelled-control conjugate 21 will bind to the control analyte 27. Hereby, a negative result is obtained being labelled both in the control line as well as the test line. In case of a positive result i.e. that haptoglobin 24 is present (shown in FIG. 2F), the haptoglobin 24 will compete with the labelled-target analyte 123 for binding to the binding molecule 125 at the test line 15. Nevertheless, the labelled-control conjugate will bind to the control analyte 27 at the control line 17. Thus, the dry stick will only be labelled at the control line 17. Depending on the concentration of haptoglobin in the raw milk, the labelled-target analyte 123 binds to the test line 15 correlating negatively (competitive) with the concentration of haptoglobin. Accordingly, the intensity of the labelling at the test line 15 will vary depending on the amount of haptoglobin present in the raw milk sample.

The labelling at the control line 17 is independent of the amount of haptoglobin present in the raw milk sample and is a control of the lateral flow as such and the release of the conjugates from the conjugate pad. If the lateral flow has run satisfactorily and the conjugates been released as should be from the conjugate pad, a labelling will appear at the control line 17. If no labelling is registered at the control line 17, the result obtained by the dry stick cannot be trusted. It is thus advantageous for the control line to be present downstream of the test line for the results at the test line to be considered valid.

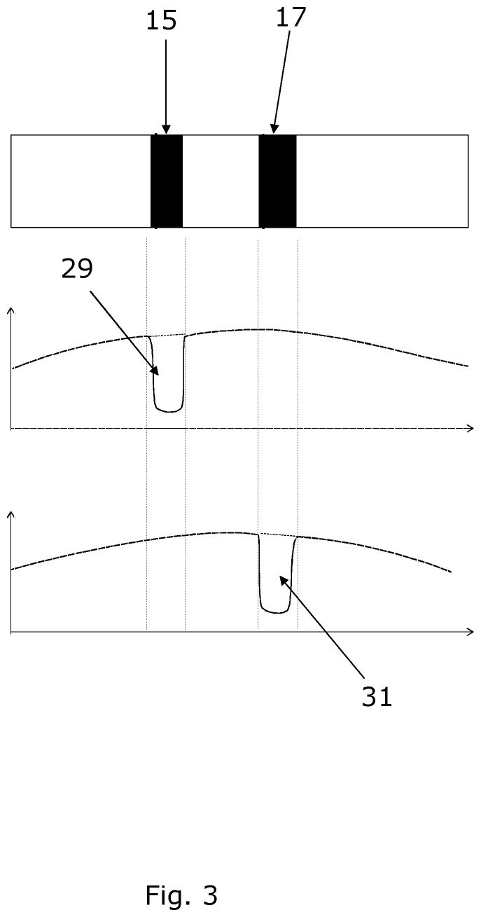

In one embodiment, the labelled conjugates are gold-labelled and the signal at the test line and control line are measured as shown in FIG. 3. This illustrates the setting of an estimated background between the vertical dotted lines based on the boundaries on either side of the test line 15 and the control line 17. The signals for each line are calculated as the integration of the area in each of the peaks 29, 31 bordered by the curved dotted topline. The peak at the test line 29 provides the test signal and the peak at the control line 31 provides the control signal. Hence, a summarized reflection number across the strip is calculated averaging the Munsell reflection value for each pixel within a defined read window as known from the Herd Navigator™ sold by DeLaval.

The test signal and the control signal is used for calculating the ratio R as R=test signal/(test signal+control signal). This ratio R is found to be essentially independent on the amounts of labelled conjugate and labelled-control conjugate released. Thus, variances in the value of haptoglobin quantity due to variance in the amount of labelled conjugate and labelled-control conjugate released can be avoided. Accordingly, potential effect of the aging of the dry stick is essentially prevented.

Reading of a dry stick may be implemented as described in e.g. WO 02/069697.

### Example 2—Dependent Vs. Independent Control Line

To increase sensitivity an independent control line was tested and compared to the results on a dry stick having a dependent control line.

The control line was immobilized with a goat anti-mouse antibody (Jackson ImmunoResearch) at a concentration of 0.3 mg/ml while the independent control line was immobilized with donkey anti-chicken antibody (Jackson ImmunoResearch) at a concentration of 0.2 mg/ml. In both cases, the test line was immobilized with haptoglobin protein at a concentration of 0.15 mg/ml.

The dry stick comprised beside the membrane with the test line and control line, a sample pad and a conjugate pad. The conjugate pad comprised labelled-conjugate and labelled-control conjugate. Both the conjugate and the control conjugate were labelled with colloidal gold according to standard procedures as commonly known to the persons skilled in the art.

The gold-labelled conjugate was a gold-labelled mouse anti-cow-antibody binding to haptoglobin, while the gold-labelled control conjugate was a gold-labelled chicken antibody (IgY). They were diffusibly arranged in a conjugate pad by soaking the conjugate pad in a solution comprising gold-labelled conjugate at a concentration of 5 μg/ml and gold-labelled control conjugate at a concentration of 10 μg/ml.

Spiked milk samples were dosed on the dry sticks with 45 μL mixed with diluent in a ratio of 45/55.

The diluent comprises water and surfactant(s), where the surfactant is around 1 w/w %. The diluent further comprises pH regulating agent(s) in the range of 0.01-0.99 w/w %.

The milk samples were spiked with haptoglobin in a concentration of 0, 1, 5 and 10 μg/ml, respectively.

The spiked milk samples were added to the dry stick and allowed to run for 5.5 minutes prior to reading the outcome of the dry stick using an optical camera as described in example 1.

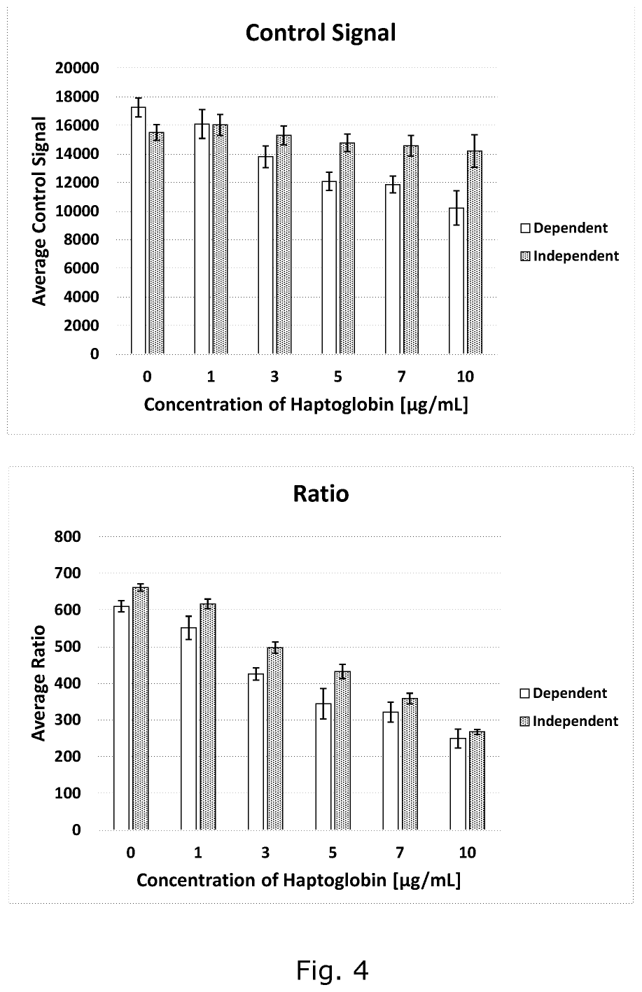

The results demonstrates that the independent control line as compared to a dependent control line shows less variation with changing haptoglobin concentration, resulting in a more stable ratio, R, value (FIG. 4). Given the relative narrow concentration range of haptoglobin in raw milk for correct cow health classification, a stabile ratio, R, value is advantageous (FIG. 4).

### Example 3—Haptoglobin Measurement in Spiked Milk Samples

A competive dry stick was used for testing of spiked milk samples. The dry stick comprised a nitrocellulose membrane, where haptoglobin protein was immobilised on the test line as a target analyte at a concentration of 0.15 mg/ml, and donkey anti-chicken antibody was immobilised on the control line as a control analyte at a concentration of 0.2 mg/ml.

Gold-labelled conjugate and labelled-control conjugate were diffusibly arranged in a conjugate pad by soaking the conjugate pad in a solution comprising gold-labelled conjugate at a concentration of 5 μg/ml and gold-labelled control conjugate at a concentration of 10 μg/ml.

The gold-labelled conjugate was a gold-labelled mouse anti-cow-antibody binding to haptoglobin, while the gold-labelled control conjugate was a gold-labelled chicken antibody (IgY). The mouse anti-cow antibody and the chicken antibody (IgY) were conjugated with gold particles using standard techniques for passive conjugation.

UHT milk samples were spiked with different concentrations of haptoglobin to test the correlation between the measurement on the dry stick and the haptoglobin concentrations. The milk was spiked with haptoglobin (Life Diagnostics) in concentrations from 0-20 μg/ml milk. 15 different concentrations were measured in 10 replicates.

The samples were mixed with diluent in a ratio of 45% milk sample and 55% diluent prior to adding the samples to the dry stick. The diluent comprises water and surfactant(s), where the surfactant is around 1 w/w %. The diluent further comprises pH regulating agent(s) in the range of 0.01-0.99 w/w %.

The spiked milk samples were added to the sample pad of the dry stick and allowed to run for 5.5 minutes prior to reading the outcome of the dry stick using an optical camera as described in example 1.

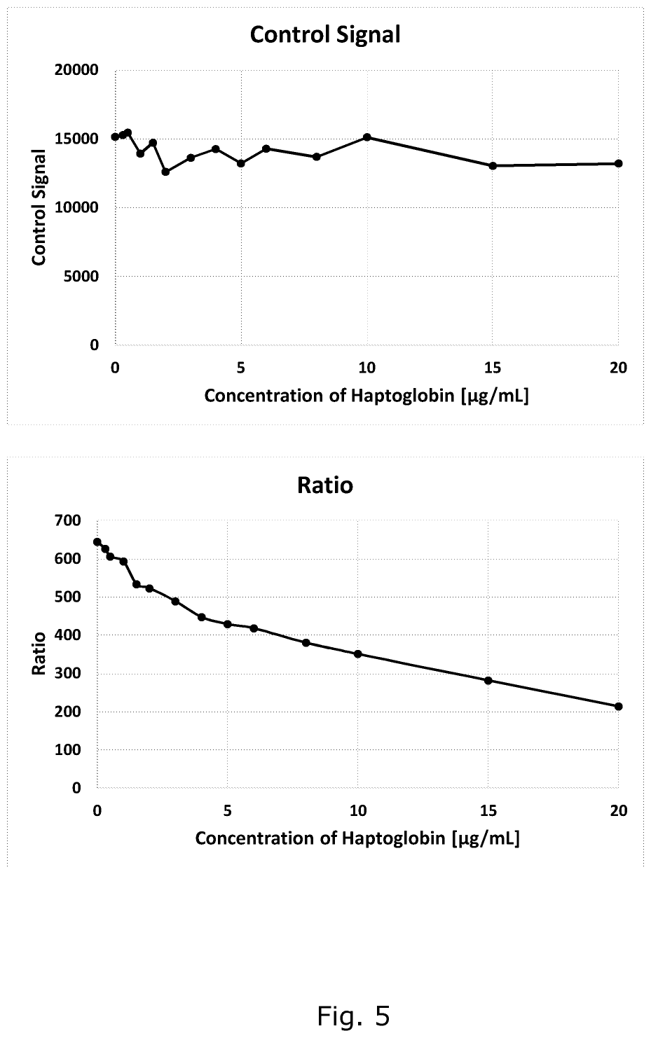

The results obtained for the measurement of the signal at the test line (TL) and the signal at the control line (CL) were used for the calculation of the ratio (R=TL/(TL+CL))(FIG. 5).

The milk samples with spiked haptoglobin showed a good sensitivity response in the concentration range 0-20 μg/ml. Response was particularly high in the 0-5 μg/ml range with a CV % significantly below 10% with an average of 4%. Saturation was not reached by 20 μg/ml haptoglobin in milk but the response was less steep from 10-20 μg/ml.

Conclusively, these data demonstrates the ability of the dry stick according to the invention to be used to measure an exact amount of haptoglobin in a milk sample as a linear relationship is observed between the values measured and the concentration of haptoglobin in the milk.

### Example 4—Haptoglobin Measurement in Farm Milk Samples

In order to test the ability of haptoglobin measurement in raw milk samples, 80 raw milk samples were obtained from an automated milking system. The samples were frozen at a maximum of 8 hours after they were obtained.

The raw milk samples were thawed for 1 hour at 37ºC prior to testing on the dry stick and visually tested for blood contamination.

The raw milk samples were added to a dry stick as described in example 4 and allowed to run for 5.5 minutes prior to reading the outcome of the dry stick using an optical camera as described in example 1.

Prior to adding the raw milk samples to the dry stick the raw milk sample was diluted with a diluent as described in example 4 in a ratio of 45% milk sample and 55% diluent.

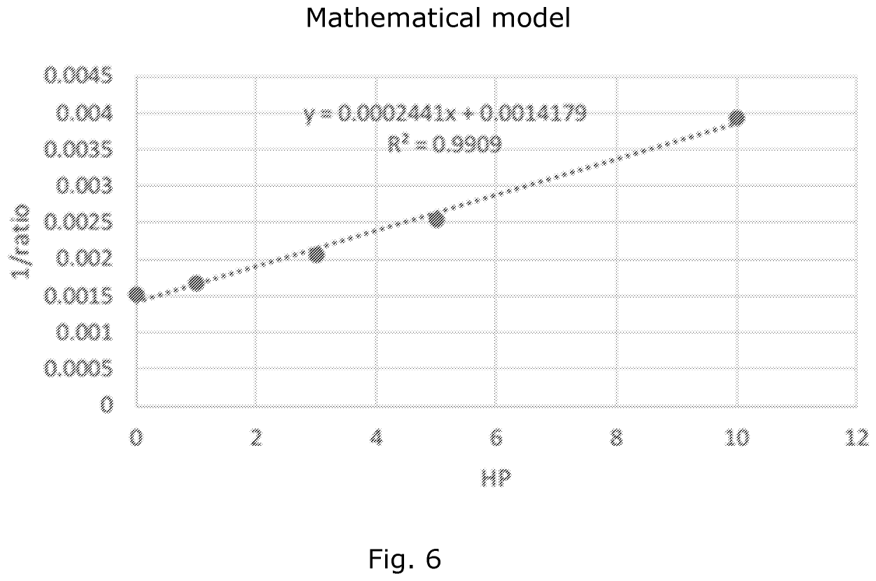

Prior to measuring the raw milk samples, a mathematical model was obtained by measuring five measurements for each of the haptoglobin concentrations 0, 1, 3, 5 and 10 μg/ml in spiked milk samples (UHT milk spiked with haptoglobin) being diluted with a diluent as described in example 4 in a ratio of 45% milk sample and 55% diluent. This resulted in a linear curve (y=0.0002441x+0.0014179; R²=0.9909) as shown in FIG. 6 when expressing 1/ratio on the y-axis and the haptoglobin concentration on the x-axis. This mathematical model was used for calculating the amount of haptoglobin for each milk sample i.e. the concentration of haptoglobin.

The values obtained by the dry stick was compared to the values obtained by using a commercially available ELISA kit (Life Diagnostics, Inc.)(data not shown). The ELISA measurements were performed following the instructions of the manufacturer.

A correlation between the values obtained by ELISA kit and by the dry stick was observed (R²=0.7820; P=7.56e-25). This demonstrates the ability of the dry stick for qualitatively measurements of the haptoglobin amount.

Conclusively, these data demonstrates that the haptoglobin level may be determined in a quantitatively manner in raw milk samples.

### Example 5—Milking Arrangement

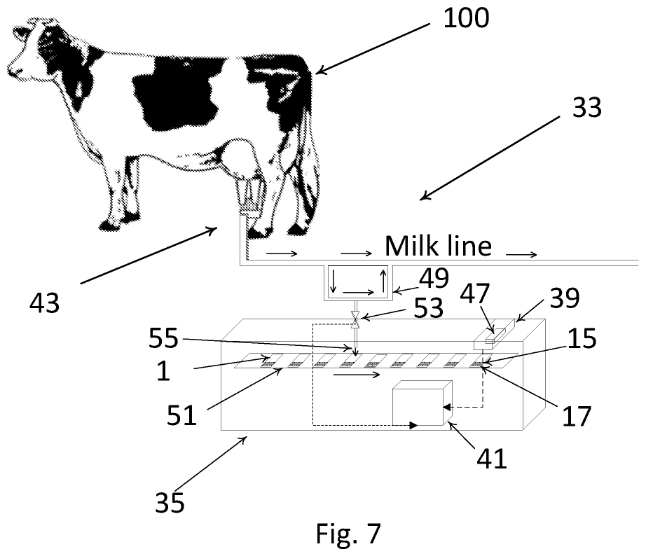

FIG. 7 schematically illustrates a milking arrangement in which the dry stick may be used. In FIG. 7, dotted lines indicates signals, such as control signal or read-out signals from sensors. As illustrated, the milking arrangement comprising milking machine 43 including teat cups attached to the udder of a cow 100 for milking milk out of the udder. The teat cups are connected to a milk line configured to feed harvest milk into a receptacle (not illustrated). A sampling line 49 is fluidicly connected to the milk line to sample milk from the milk flowing in the milk line. In FIG. 7, the sampling line 49 is illustrated as bypass line with an electronically controlled valve 53. The sampling line 49, valve 53 and outlet forms at least part of a sampling device 45.

The milking arrangement further comprises a conveyer 51 on which sticks 1 are placed and the conveyer conveys the sticks in a direction from left to right relatively to the orientation of FIG. 7.

An outlet 55 downstream of the valve 53 is arranged in close proximity to the conveyer 53 so that when a stick is conveyed to a position below the outlet 55, opening of the valve 53 provides one or more drops of milk to be applied to the stick 1. In a position away from the outlet 55, a haptoglobin sensor device 39 is arranged. In the disclosed embodiment, the haptoglobin sensor device 39 has an optical reader comprising a CCD camera device, which is arranged to optically read the test signal, and if implemented, the control signal produced by the stick.

The milking arrangement comprises a processor 41. The processor 41 is configured convert the signal(s) from the stick 1 (received from the haptoglobin sensor device 39 into a value of haptoglobin quantity in a milk sample. The processor 41 receives the signal from the sensor 39 typically as electrical signals as illustrated by the dotted lines in FIG. 7. The processor 41 is furthermore configured to control opening and closing of the valve 53 to allow milk to be applied to the stick 1. In addition, the processor is furthermore configured to control the movement of the conveyer 51 to advance sticks located on the conveyer 51 towards the outlet 55 providing milk and towards the haptoglobin sensor device 39.

As can be readily understood, providing a distance between the position where the milk is applied to the stick and where the signal(s) produced by the stick is obtained by the haptoglobin sensor device 39, represents a time during which the signal(s) can evolve on the stick(s). This time can be controlled by controlling the conveying speed, whereby the time during which the signal(s) evolves is controllable by the conveying speed. Thereby, if a certain amount of reaction time is aimed at, this may be accomplished by setting the conveying speed accordingly. However, it is to be emphasised that other ways of controlling the reaction time may be used.

In the disclosed embodiment of FIG. 7, the conveyer 51, the controller 41 and the haptoglobin sensor device 39 are illustrated as being contained in a physical delimited analysing unit 35, where the physical delimit is provided by a cabinet. By this, the interior of the cabinet may form an incubator where the atmosphere, such as humidity and/or temperature can be controlled.

Although the present invention has been described in connection with the specified embodiments, it should not be construed as being in any way limited to the presented examples. The scope of the present invention is set out by the accompanying claim set. In the context of the claims, the terms “comprising” or “comprises” do not exclude other possible elements or steps. Also, the mentioning of references such as “a” or “an” etc. should not be construed as excluding a plurality. The use of reference signs in the claims with respect to elements indicated in the figures shall also not be construed as limiting the scope of the invention. Furthermore, individual features mentioned in different claims, may possibly be advantageously combined, and the mentioning of these features in different claims does not exclude that a combination of features is not possible and advantageous.

### REFERENCE NUMBERS

1: Dry stick
3: Backing card
5: Sample pad
8: Reagent pad
9: Conjugate pad
11 Membrane
13 Absorbent pad
15: Test line
17: Control line
19: Direction of lateral flow
21: Labelled-control conjugate
23: Labelled-conjugate
24 Haptoglobin
25: Target analyte
27 Control analyte
29 Peak
31: Peak
33: Milking arrangement
35: Analysing unit
39: Haptoglobin sensor device
41: Processor
43: Milking machine
45: Sampling device
47: CCD
49 Sampling line
51 Conveyor
53: Valve
55: Outlet
100: Cow
123: Labelled-target analyte
125: Binding molecule

1. A dry stick being a competitive lateral flow stick configured to measure haptoglobin in a raw milk sample, said dry stick comprising:
a base pad that allows lateral flow of fluid there through,
the base pad comprising a labelled-control conjugate and a labelled-conjugate diffusibly arranged herein,
wherein said labelled-conjugate binds haptoglobin, and wherein a complex is formed between said labelled-conjugate and said haptoglobin when said dry stick is in use and binds with haptoglobin;
wherein said base pad further comprises
a test line comprising immobilised target analyte, wherein said immobilised target analyte binds to said labelled-conjugate when not in said complex; and
a control line, the control line being spaced from said test line, and wherein the control line comprises control analyte that binds to said labelled-control conjugate.

2. A dry stick being a competitive lateral flow stick configured to measure haptoglobin in a raw milk sample, said dry stick comprising:
a base pad that allows lateral flow of fluid there through,
the base pad comprising a labelled-control conjugate and a labelled-target analyte diffusibly arranged herein;
wherein said base pad further comprises
a test line comprising an immobilised binding molecule, wherein said immobilised binding molecule binds to haptoglobin or said labelled-target analyte; and
a control line, the control line being spaced from said test line, and wherein the control line comprises a control analyte that binds to said labelled-control conjugate.

3. The dry stick according to claim 1, wherein said base pad further comprises a surfactant.

4. The dry stick according to claim 3, wherein said surfactant is Pluronic F68, Pluronic F127, Surfactant 10G, Synperonic F108, Tergitol, Tween-20 and/or Triton X-100.

5. The dry stick according to claim 3, wherein said surfactant is in a concentration of 0.1-5%.

6. The dry stick according to claim 3, wherein said base pad comprises at least two modules, wherein one of said at least two modules is a sample pad configured for receiving said raw milk sample and wherein said sample pad comprises said surfactant.

7. The dry stick according to claim 1, wherein said control line is downstream of said test line.

8. The dry stick according to claim 1, wherein said base pad comprises a membrane; said membrane comprising said test line and said control line.

9. The dry stick according to claim 8, wherein said membrane is a nitrocellulose membrane.

10. The dry stick according to claim 1, wherein said labelled-conjugate is a first antibody.

11. The dry stick according to claim 10, wherein said labelled-control conjugate is a second antibody.

12. The dry stick according to claim 11, wherein said control analyte is a third antibody.

13. The dry stick according to claim 1, wherein said labelled-control conjugate and/or said labelled-conjugate comprises a label selected from the group consisting of: colloidal gold particles, latex particles, cellulose nanobeads, paramagnetic particles, radioactive particles, fluorescent particles, and enzymes.

14. The dry stick according to claim 13, wherein said label is colloidal gold particles.

15. The dry stick according to claim 1, wherein said base pad comprises a pH-regulating agent.

16. The dry stick according to claim 15, wherein said pH-regulating agent is a phosphate buffer, a borate buffer, a citric acid buffer and/or a Tris buffer.

17. A method of measuring haptoglobin in a raw milk sample, comprising the steps of:
a) providing an raw milk sample potentially comprising haptoglobin;
providing the dry stick of claim 1;
c) applying said raw milk sample to said dry stick;
d) allowing said raw milk sample to flow through said test line and said control line on said dry stick; and
e) determining the amount of haptoglobin from said test line and from said control line.

18. The method according to claim 17, wherein said raw milk sample is provided in a defined amount.

19. The method according to claim 17, wherein said raw milk sample is mixed with a diluent comprising a surfactant, prior to applying said raw milk sample to said dry stick.

20. The method according to claim 19, wherein said surfactant is present in the diluent in a concentration of 0.1-4 w/w %.

21. The method according to claim 19, wherein said surfactant is one or more surfactants selected from the group consisting of: Pluronic F68, Pluronic F127, Surfactant 10G, Synperonic F108, Tergitol, Tween-20, and/or Triton X-100.

22. The method according to claim 21, wherein said surfactant is Tween-20.

23. The method according to claim 17, wherein said amount of haptoglobin is determined by measuring a test signal at said test line and a control signal at said control line, and calculating a signal ratio by dividing said test signal with a sum of said test signal and said control signal, and following using said signal ratio to retrieve said amount of haptoglobin from a look-up table or by applying a mathematical model.

24. The method according to claim 23, wherein said test signal and/or control signal is measured using an optical device.
